(19) United States
(12) Patent Application Publication
(10) Pub. No.: US 2010/0222394 A1
(43) Pub. Date: Sep. 2, 2010

(54) METHOD FOR PRODUCING PYRAZOL-3-YL-BENZAMIDE DERIVATIVE

(76) Inventors: Kenichi Asakawa, Shiga (JP); Naotaka Sawada, Osaka (JP); Takayuki Tsuritani, Nara (JP); Toshiaki Mase, Ibaraki (JP); Keiji Takahashi, Chiba (JP); Takahiro Itoh, Fanwood, NJ (US); Feng Xu, Staten Island, NY (US); Naoki Yoshikawa, Edison, NJ (US)

Correspondence Address: MERCK, P O BOX 2000, RAHWAY, NJ, 07065-0907, US

(21) Appl. No.: 12/679,836

(22) PCT Filed: Sep. 25, 2008
(86) PCT No.: PCT/JP2008/067254

§ 371 Date: May 7, 2010

Related U.S. Application Data
(60) Provisional application No. 61/190,865, filed on Sep. 3, 2008.

(30) Foreign Application Priority Data
Sep. 28, 2007 (JP) 2007-253602

(51) Int. Cl. A61K 31/4439; C07D 401/12; C07D 213/71; A61P 3/10; A61P 3/04

(52) U.S. Cl. 514/341; 546/275.4; 546/294

(57) ABSTRACT

The present invention provides a more efficient industrial method for producing a pyrazol-3-yl-benzamide derivative expressed by a formula useful as medicine:

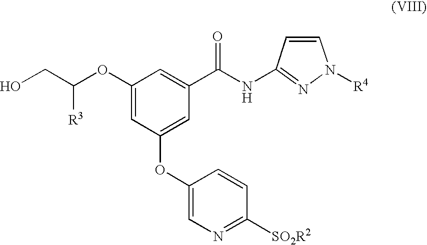

wherein R², R³ and R⁴ each independently represent a lower alkyl group.

### FIELD OF THE INVENTION

The present invention relates to a more efficient and new production method for a pyrazol-3-yl-benzamide derivative useful as medical products. Further, it also relates to an intermediate for producing the pyrazol-3-yl-benzamide derivative efficiently. And it relates to crystalline of pyrazol-3-yl-benzamide derivative which is useful as medicine.

### DESCRIPTION OF THE RELATED ART

A pyrazol-3-yl-benzamide derivative has a strong activating action of glucokinase (hereinafter, also called GK), and it is known useful as a therapeutic substance and/or a preventive substance of diabetes, or a therapeutic and/or preventive substance for chronic complication of diabetes such as retinopathy, nephropathy, neurosis, ischemic heart disease and arterial sclerosis, further as a therapeutic and/or preventive substance of obesity (see Patent document 1).

[Patent document 1]

### WO2004/076420

### DISCLOSURE OF THE INVENTION

### Problems that the Invention is to Solve

In the production method for a pyrazol-3-yl-benzamide derivative shown in Patent document 1, the number of processes is many, and purification of an intermediate is done only by purification through column, which has been the point to be improved regarding an efficient production method for a pyrazol-3-yl-benzamide derivative and its impurity. And in aforementioned Patent document 1, any salt of pyrazole-3-yl-benzamide derivative is not described.

The present inventors have keenly studied to develop a more efficient industrial method for producing a pyrazol-3-yl-benzamide derivative than the method for producing a pyrazol-3-yl-benzamide derivative shown in Patent document 1, as a result, found a new production method for a pyrazol-3-yl-benzamide derivative and its salt satisfying the points of efficiency and purity by decreasing the number of processes and isolating an intermediate as a salt, and completed the present invention.

Namely, the present invention relates to production methods of (1) to (5), and new compounds of (6) to (17) below, and a pharmaceutical composition comprising an alky sulfonate of a pyrazol-3-yl-benzamide derivative as an active ingredient.

(1) A method for producing a compound expressed by a formula (VIII) or a pharmaceutically acceptable salt thereof, comprising the steps of:

reacting a compound expressed by a formula (II) in the presence of compound represented by the formula (I) and base,

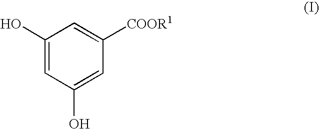

wherein R¹ represents a lower alkyl group,

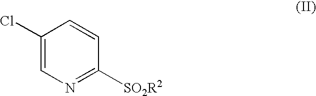

wherein R² represents a lower alkyl group;

reacting a resulting compound expressed by a formula (III) with a compound expressed by a formula (IV) in the presence of base,

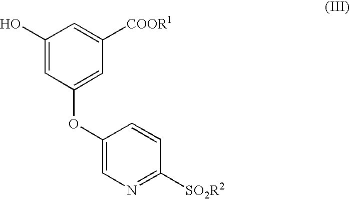

wherein R¹ and R² have the same meaning as described above,

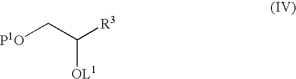

wherein P¹ represents a protective group of a hydroxyl group, R³ represents a lower alkyl group, and OL₁ represents a leaving group;

removing a protective group P¹ of a hydroxyl group and a protecting group R¹ of a carboxyl group in a resulting compound expressed by a formula (V),

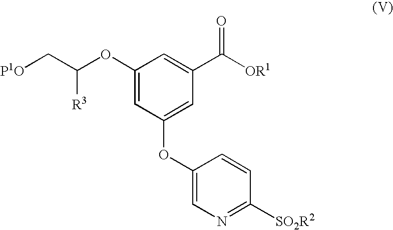

wherein R¹, R², R³ and P¹ have the same meaning as described above;

reacting a resulting compound expressed by a formula (VI) with an amine,

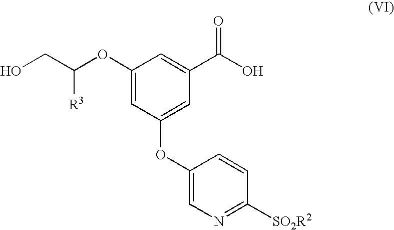

wherein R² and R³ have the same meaning as described above;

after producing a salt containing a carboxylic acid derivative expressed by the formula (VI) and an amine of 2:1, condensing said salt with a primary amine compound expressed by a formula (VII),

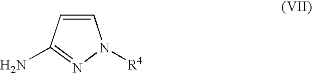

wherein R⁴ represents a lower alkyl group, to produce the compound expressed by a formula (VIII),

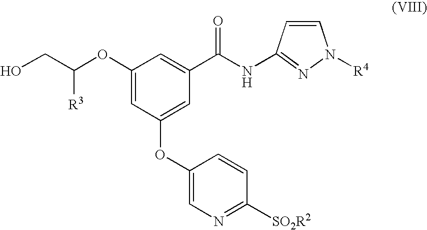

wherein R², R³ and R⁴ have the same meaning as described above.

(2) A method for producing a compound expressed by a formula (VIII)

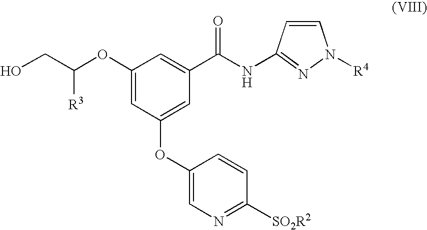

wherein R², R³ and R⁴ have the same meaning as described above, or a pharmaceutically acceptable salt thereof, comprising the step of condensing a salt containing a compound expressed by a formula (VI) and an amine of 2:1 with a primary amine compound expressed by a formula (VII),

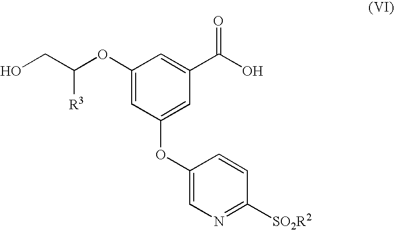

wherein R² and R³ represent a lower alkyl group,

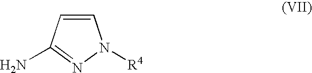

wherein R⁴ represents a lower alkyl group.

(3) The production method described in (1) or (2), wherein the pharmaceutically acceptable salt of a compound expressed by the formula (VIII) is a methanesulfonate.

(4) The production method of any one described in (1) to (3), wherein the amine is 1,4-diazabicyclo[2.2.2]octane.

(5) The production method of any one described in (1) to (4), wherein R² is an ethyl group, R³ and R⁴ are a methyl group.

(6) A 1,4-diazabicyclo[2.2.2]octane ½ salt of a compound expressed by a formula (VI):

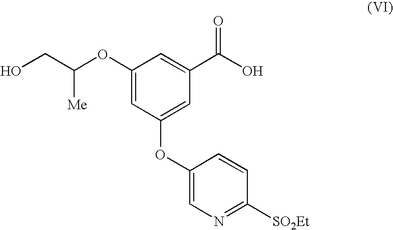

(7) An alkylsulfonate of a compound expressed by a formula (VIII):

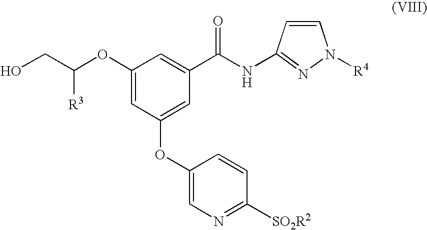

wherein R², R³ and R⁴ represents a lower alkyl group.

(8) A methanesulfonate of a compound expressed by a formula (VIII):

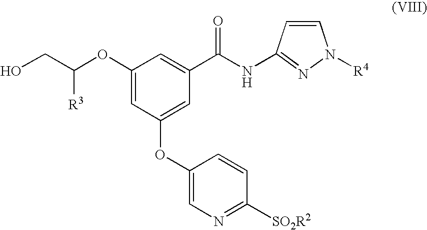

wherein R², R³ and R⁴ represent a lower alkyl group.

(9) A methanesulfonate of a compound expressed by a formula (VIII-1):

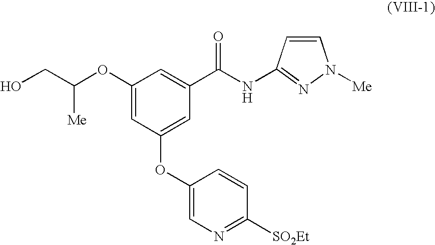

(10) A crystalline form of 3-(6-ethanesulfonylpyridin-3-yloxy)-5-((1S)-2-hydroxy-1-methyl-ethoxy)-N-(1-methyl-1H-pyrazol-3-yl)benzamide methanesulfonate.

(11) A crystalline form of 3-(6-ethanesulfonylpyridin-3-yloxy)-5-((1S)-2-hydroxy-1-methyl-ethoxy)-N-(1-methyl-1H-pyrazol-3-yl)benzamide methanesulfonate having main peaks at around 9.6, 11.8, 18.8, 19.2, 19.7, 20.3, 21.3, 21.8 and 23.7 in terms of 2θ(°) in the powder X-ray diffraction.

(12) A crystalline form of 3-(6-ethanesulfonylpyridin-3-yloxy)-5-((1S)-2-hydroxy-1-methyl-ethoxy)-N-(1-methyl-1H-pyrazol-3-yl)benzamide methanesulfonate having T onset at 137° C. and T max at 140° C. and heat of fusion is 106 J/g in the DSC analysis.

(13) A crystalline form of 3-(6-ethanesulfonylpyridin-3-yloxy)-5-((1S)-2-hydroxy-1-methyl-ethoxy)-N-(1-methyl-1H-pyrazol-3-yl)benzamide methanesulfonate having main peaks at around 9.6, 11.8, 18.8, 19.2, 19.7, 20.3, 21.3, 21.8 and 23.7 in terms of 2θ(°) in the powder X-ray diffraction and having endothermic peak at 137-140° C. in the DSC analysis.

(14) A crystalline form according to any one of claims 10 to 13, characterized by the following absorptions in the FT-IR spectrum (KBr pellet-transmission method): 3355, 3112, 1602, 1567, 1311, 1225, 1205, 1164 and 779 cm⁻¹.

(15) A pharmaceutical composition comprising any one of the crystalline of above (10) to (14).

(16) A glucokinase activator comprising any one of the crystalline of above (10) to (14).

(17) An agent for treating diabetes comprising any one of the crystalline of above (10) to (14).

### BEST MODE FOR CARRYING OUT THE INVENTION

A lower alkyl group represented by R¹ means a linear or branched alkyl group having carbon numbers of 1 to 6, specifically, for example, there are listed a methyl group, ethyl group, propyl group, isopropyl group, n-butyl group and the like, above all, a methyl group or an ethyl group is preferable.

A lower alkyl group represented by R² means a linear or branched alkyl group having carbon numbers of 1 to 6, specifically, for example, there are listed a methyl group, ethyl group, propyl group, isopropyl group, n-butyl group and the like, above all, a methyl group or an ethyl group is preferable, and an ethyl group is more preferable.

A lower alkyl group represented by R³ means a linear or branched alkyl group having carbon numbers of 1 to 6, specifically, for example, there are listed a methyl group, ethyl group, propyl group, isopropyl group, n-butyl group and the like, above all, a methyl group or an ethyl group is preferable, and a methyl group is more preferable.

A lower alkyl group represented by R⁴ means a linear or branched alkyl group having carbon numbers of 1 to 6, specifically, for example, there are listed a methyl group, ethyl group, propyl group, isopropyl group, n-butyl group and the like, above all, a methyl group or an ethyl group is preferable, and a methyl group is more preferable.

A protective group of a hydroxyl group represented by R¹ mentions, for example, a protective group of a hydroxyl group described in Protective Groups in Organic Synthesis (written by T. W. Green, second edition, published by John Wiley & Sons, 1991) etc., and specifically, for example, tert-butyldimethylsilyl group or the like is listed.

As a leaving group represented by OL¹, for example, an alkylsulfonyloxy group or an arylsulfonyloxy group and the like are listed, specifically, there are listed a methanesulfonyloxy group, ethanesulfonyloxy group, benzenesulfonyloxy group, p-toluenesulfonyloxy group and the like.

Hereinafter, a production method for the pyrazol-3-yl-benzamide derivative of the present invention will be described.

### Production of Compound (III)

By reacting a compound expressed by a formula (I) with a compound expressed by a formula (II), a compound expressed by a formula (III) can be produced.

A compound expressed by the formula (I) is simply called a compound (I). Further, a compound expressed by the formula (II) is simply called a compound (II).

The reaction of compound (I) and compound (II) is conducted ordinarily in a solvent in the presence of base. As the solvent, any one may be used as long as it does not hinder the reaction of compound (I) and compound (II), for example, an amide type solvent is mentioned, more specifically, for example, preferable are N-methyl-2-pyrrolidinone, N,N-dimethylformamide, 1,3-dimethyl-2-imidazolidinone and N,N-dimethylacetoamide (DMAc), above all, N,N-dimethylacetoamide is more preferable. These solvents can be used alone or in a mixed solvent of 2 kinds or more thereof.

As the base, for example, there are listed tert-butoxide of alkali metal such as potassium—tert-butoxide and sodium—tert-butoxide, and alkoxide of alkali metal such as sodium methoxide and sodium ethoxide. Other bases such as alkaline carbonates also work for this transformation. Above all, tert-butoxide of alkali metal is preferable, and particularly, potassium tert-butoxide is preferable.

The amount of base used is 1 to 10 equivalents relative to 1 equivalent of compound (I), and preferably 1 to 3 equivalents.

The amount of compound (II) used is ordinarily 0.4 to 5 equivalents relative to 1 equivalent of compound (I), and preferably 0.5 to 1 equivalents.

The reaction temperature is ordinarily 0° C. to 150° C., and preferably 50° C. to 120° C.

The reaction time is ordinarily 10 minutes to 12 hours, and preferably 30 minutes to 10 hours.

### Production of Compound (V)

By reacting a compound expressed by a formula (III) with a compound expressed by a formula (IV) (hereinafter called a compound (IV)), a compound expressed by a formula (V) (hereinafter called a compound (V)) can be produced.

The reaction of compound (III) and compound (IV) is conducted ordinarily in a solvent in the presence of base. As the solvent, any one may be used as long as it does not hinder the reaction of compound (III) and compound (IV), for example, an amide type solvent is mentioned, more specifically, for example, preferable are N-methyl-2-pyrrolidinone, N,N-dimethylformamide, 1,3-dimethyl-2-imidazolidinone and N,N-dimethylacetoamide (DMAc), above all, N,N-dimethylacetoamide is more preferable. These solvents can be used alone or in a mixed solvent of 2 kinds or more thereof.

As the base, preferable are sodium carbonate, potassium carbonate, cesium carbonate, triethylamine, diisopropylethylamine and the like, and cesium carbonate is more preferable.

The amount of base used is 0.5 to 10 equivalents relative to 1 equivalent of compound (III), and preferably 1 to 5 equivalents.

The amount of compound (IV) used is ordinarily 1 to 10 equivalents relative to 1 equivalent of compound (III), and preferably 1 to 5 equivalents.

The reaction temperature is ordinarily 0° C. to 150° C., and preferably 20° C. to 100° C.

The reaction time is ordinarily 10 minutes to 12 hours, and preferably 30 minutes to 6 hours.

### Production of Compound (VI)

By removing a protective group P¹ of a hydroxyl group and a protecting R¹ of a carboxyl group in the compound (V), a compound (VI) can be produced.

A compound expressed by the formula (VI) is simply called a compound (VI).

The removal of a protective group P¹ of a hydroxyl group can be done by a method described in Protective Groups in Organic Synthesis (written by T. W. Green, second edition, published by John Wiley & Sons, 1991) etc., a method based thereon, or a combination method of these and an ordinary method, for example, by reacting the compound (V) with hydrochloric acid in a solvent such as tetrahydrofuran (hereinafter called THF) and methanol, or a mixed solvent thereof, a protective group P¹ of a hydroxyl group can be removed, subsequently, for example, by reacting a compound that a protective group P¹ of a hydroxyl group in the compound (V) was removed and sodium hydroxide in a solvent such as THF and methanol, or a mixed solvent thereof, a compound (VI) can be produced.

The amount of hydrochloric acid used is ordinarily 0.5 to 20 equivalents relative to 1 equivalent of compound (V), and preferably 1 to 10 equivalents.

The amount of sodium hydroxide used is ordinarily 0.5 to 20 equivalents relative to 1 equivalent of compound (V), and preferably 1 to 10 equivalents.

The reaction temperature is ordinarily 0° C. to 100° C., and preferably 20° C. to 50° C.

The reaction time is ordinarily 10 minutes to 12 hours, and preferably 30 minutes to 5 hours.

Removal of carboxy protecting group followed by removal of hydroxyl protecting group, also can produce the compound of formula (VI).

Production of Salt of Compound (VI) with Amine

By reacting the compound (VI) with an amine, a salt of the compound (VI) with amine can be produced.

The reaction of compound (VI) and amine is conducted ordinarily in a solvent. As the solvent, there are listed esters (for example, ethyl acetate, methyl acetate, isopropyl acetate, etc.), and alcohols (methanol, ethanol). These solvents can be used alone or in a mixed solvent of 2 kinds or more thereof.

As the amine, for example, diisopropyl amine, diisopropylhexyl amine, 1,4-diazabicycho[2.2.2]octane (hereinafter, also called DABCO) are listed and DABCO is preferable.

Since a salt of the compound (VI) with amine can be isolated, a production method for a pyrazol-3-yl-benzamide derivative related to the present invention is suitable for a more industrial production method than the conventional production method particularly in the point of purity.

The amount of the cyclic diamine is ordinarily 0.1 to 5 equivalents relative to 1 equivalent of compound (VI), and preferably 0.2 to 2 equivalents.

The reaction temperature is ordinarily 0° C. to 100° C., and preferably 20° C. to 70° C.

The reaction time is ordinarily 1 hour to 2 days, and preferably 5 hours to 1 day.

A salt of the compound (VI) with amine may also be recrystallized for use. As a solvent used in recrystallization, for example, alcohols solvent (for example, methanol ethanol, etc.) are listed, and ethanol is preferable.

In crystallization or recrystallization, seed crystal can be used.

### Production of Compound (VIII)

By reacting the salt of compound (VI) with amine and a compound expressed by a formula (VII), a compound expressed by a formula (VIII) can be produced.

A compound expressed by the formula (VII) is simply called a compound (VII), and, a compound expressed by the formula (VIII) is simply called a compound (VIII).

The reaction of the salt of compound (VI) with amine and a compound (VII) is conducted ordinarily in a solvent. As the solvent, any one may be used as long as it does not interfere the reaction, for example, there are listed methylene chloride, chloroform, 1,2-dichloroethane, dimethylformamide, acetic acid ethyl ester, acetic acid methyl ester, acetonitrile, benzene, xylene, toluene, 1,4-dioxane, tetrahydrofuran, dimethoxyethane, water or a mixed solvent thereof, above all, a mixed solvent of acetonitrile with water is preferable.

Regarding the reaction of a salt of compound (VI) with amine and a compound (VII), an ordinary amide-forming reaction may be conducted by a method described in documents (for example, “Basis and Experiments of Peptide Synthesis” (written by Izumiya Nobuo et. al, published by Maruzen Co., Ltd., 1983), “Comprehensive Organic Synthesis” (sixth volume, published by Pergamon Press Corporation, 1991) etc), a method based thereon, or a combination method of these and an ordinary method, namely, it can be conducted by using a condensation agent known to those in the art, or by an ester activation method usable to those in the art, a mixed acid anhydride method, an acid chloride method, a carbodiimide method and the like. As such amide-forming reagent, for example, there are listed, thionyl chloride, oxalyl chloride, N,N-dicyclohexylcarbodiimide, N,N′-carbonyldiimidazole, diphenylphosphoryl chloride, N,N′-disuccinimidyl carbonate, N,N′-disuccinimidyl oxalate, 1-ethyl-3-(3-dimethylaminopropyl)carbodiimide hydrochloride, ethyl chloroformate, isopropyl chloroformate and the like, above all, for example, 1-ethyl-3-(3-dimethylaminopropyl)carbodiimide hydrochloride is preferable. Further, in an amide-forming reaction, base and condensation auxiliaries may be used together with the above-described amide-forming reagent.

As the base used, for example, there are listed tertiary fatty amines such as trimethylamine, triethylamine, N,N-diisopropylethylamine, N-methylmorpholine, N-methylpyrrolidine, N-methylpiperidine, N,N-dimethylaniline, 1,8-diazabicyclo[5.4.0]undeca-7-ene (DBU), 1,5-diazabicyclo[4.3.0]nona-5-ene (DBN); for example, aromatic amines such as pyridine, 4-dimethylaminopyridene, picoline, lutidine, quinoline or isoqunoline, above all, pyridine is preferable.

As the condensation auxiliary, for example, there are listed N-hydroxybenzotriazole hydrate, N-hydroxysuccinimide, N-hydroxy-5-norbornene-2,3-dicarboxylmide, or 3-hydroxy-3,4-dihydro-4-oxo-1,2,3-benzotriazole and the like.

The amount of compound (VII) used is ordinarily 0.5 to 10 equivalents relative to 1 equivalent of the salt of compound (VI) and cyclic diamine, and preferably 1 to 5 equivalents.

The amount of amide-forming reagent used is ordinarily 0.5 to 10 equivalents, and preferably 1 to 5 equivalents.

The amount of base used is ordinarily 0.5 to 10 equivalents, and preferably 1 to 5 equivalents.

The amount of condensation auxiliary used is ordinarily 0.5 to 10 equivalents, and preferably 1 to 5 equivalents.

The reaction time is ordinarily 0.5 hours to 24 hours, and preferably 0.5 to 5 hours.

The reaction temperature is ordinarily 0° C. to 100° C., and preferably 0° C. to 50° C.

Production of Salt of Compound (VIII) with Alkylsulfonic Acid

By reacting the compound (VIII) with an alkylsulfonic acid, a salt of the compound (VIII) and alkylsulfonic acid can be produced.

The reaction of compound (VIII) and alkylsulfonic acid is conducted ordinarily in a solvent. As the solvent, any one may be used as long as it does not interfere the reaction, for example, there are listed methylene chloride, chloroform, 1,2-dichloroethane, dimethylformamide, acetic acid ethyl ester, acetic acid methyl ester, acetonitrile, benzene, xylene, toluene, 1,4-dioxane, tetrahydrofuran, dimethoxyethane, or a mixed solvent thereof, above all, a mixed solvent of acetonitrile with toluene is preferable.

As the alkylsulfonic acid used, methansulfonic acid, ethanesulfonic acid and the like are listed.

The amount of alkylsulfonic acid used is ordinarily 0.5 to 5 equivalents relative to 1 equivalent of compound (VIII), and preferably 1 to 3 equivalents.

The reaction temperature is ordinarily 0° C. to 80° C., and preferably 20° C. to 60° C.

The reaction time is ordinarily 1 to 48 hours, and preferably 5 to 20 hours.

Among the alkylsulfonate of the present invention of formula (VIII) which is useful as drugs for treating and/or preventing diabetes, methanesulfonate of formula (VIII) is preferable, and methanesulfonate of formula (VIII-1) is more preferable.

A crystalline form of 3-(6-ethanesulfonylpyridin-3-yloxy)-5-((1S)-2-hydroxy-1-methyl-ethoxy)-N-(1-methyl-1H-pyrazol-3-yl)benzamide methanesulfonate means the crystalline having main peaks at around 9.6, 11.8, 18.8, 19.2, 19.7, 20.3, 21.3, 21.8 and 23.7 in terms of 2θ(°) in the powder X-ray diffraction.

The diffraction peak in 2θ(°) have some measurement error due to measuring instrument or measuring conditions. The measurement error may be within the range of ±0.2, preferably ±0.1.

The crystalline of 3-(6-ethanesulfonylpyridin-3-yloxy)-5-((1S)-2-hydroxy-1-methyl-ethoxy)-N-(1-methyl-1H-pyrazol-3-yl)benzamide methanesulfonate can be also characterized by thermoanalysis, Said crystalline have Tonset at 137° C., T max at 140° C. in the DSC (Differential Scanning Calorimetry) analysis, and heat of fusion (ΔH) at 106 J/g. T max means endothermic peak. Tonset means a temperature of intersection with the tangent and baseline, wherein the tangent is pulled so as to maximize the integration value of a low temperature side of endothermic curve of DSC peak.

The crystalline of 3-(6-ethanesulfonylpyridin-3-yloxy)-5-((1S)-2-hydroxy-1-methyl-ethoxy)-N-(1-methyl-1H-pyrazol-3-yl)benzamide methanesulfonate can be also characterized by infrared absorption spectrum. The infrared absorption spectrum of said crystal is as table 1.

TABLE 1

| wave number | |
|---|---|
| 3355 (br) | O—H stretching |
| 3112 | N—H stretching |
| 1602 | Carbony C═O stretching (aromatic amide) |
| 1567 | C═C stretching |
| 1311, 1225 | Aromatic ether |
| 1205 | S═O stretching |
| 1164 | C—O stretching |
| 779 | Aromatic C—H antiplane bending vibration |

The crystalline of 3-(6-ethanesulfonylpyridin-3-yloxy)-5-((1S)-2-hydroxy-1-methyl-ethoxy)-N-(1-methyl-1H-pyrazol-3-yl)benzamide methanesulfonate can be used as active ingredient for treating and/or preventing diabetes, for treating and/or preventing diabetes chronic complication such as retinopathy, nephropathy, neurosis, ischemic cardiac disease and arterial sclerosis, for treating and/or preventing obesity.

Depending on the type of the substituents therein, the compounds of the invention include stereoisomers and tautomers such as optical isomers, diastereomeric isomers and geometrical isomers. Needless-to-say, the compounds of the invention include all these isomers. Further needless-to-say, the compounds of the invention include all mixtures of such isomers.

In producing medicines for prevention and remedy for type II diabetes or diseases or symptoms associated with it, the alkylsulfonate of the compound of (VIII), methanesulfonate of the compound of (VIII), or methanesulfonate of (VIII-1) may be combined with carrier.

The alkylsulfonate of the compound of (VIII), methanesulfonate of the compound of (VIII), or methanesulfonate of (VIII-1) is also referred to as a compound of aforementioned formula (VIII) etc.

Among the alkylsulfonate of formula (VIII), methanesulfonate of formula (VIII) and methanesulfonate of formula (VIII-1), the methanesulfonate of formula (VIII-1) and/or its crystalline is preferable.

The dose of the compounds of formula (VIII) of the invention for prevention or remedy for diseases naturally varies, depending on the property of the symptom to which the treatment is directed, the specific compound selected for it and the administration route.

The dose also varies depending on the age, the body weight and the sensitivity of patients.

In general, the daily dose for one-time or plural-times administration may be from about 0.001 mg/kg-body weight to about 100 mg/kg-body weight, preferably from about 0.01 mg/kg-body weight to about 50 mg/kg-body weight, even more preferably from about 0.1 mg/kg-body weight to about 10 mg/kg-body weight. As the case may be, administration of a dose over the range may be necessary.

An example of a suitable dose for oral administration is described. The daily dose for one-time or two- to four-times administration may be at least from about 0.01 mg to at most 2.0 g. Preferably, the daily administration frequency is once or twice a day, and the daily dose is from about 1.0 mg to about 200 mg. More preferably, the daily dose is from about 10 mg to 100 mg for one-time administration a day.

For intravenous administration or oral administration, a typical dose of the compound (VIII) may be from about 0.001 mg/day/kg-body weight to about 100 mg/day/kg-body weight (preferably from 0.01 mg/day/kg-body weight to about 10 mg/day/kg-body weight), more preferably from about 0.1 mg/day/kg-body weight to 10 mg/day/kg-body weight.

As so mentioned hereinabove, the pharmaceutical composition of the invention comprises a compound of formula (VIII) and a pharmaceutically-acceptable carrier. The term “composition” is meant to contain not only a product produced by directly or indirectly combining, hybridizing or aggregating 2 or more ingredients, a product produced as a result of dissociation of one or more ingredients, or a compound produced as a result of reaction or interaction of different types of ingredients, but also an active and inactive ingredient of constituting a carrier (pharmaceutically-acceptable vehicle).

As combined with a pharmaceutically-acceptable carrier, the composition of the invention preferably contains a compound of formula (VIII) in an amount effective for remedy and prevention of type II diabetes and for retardation of the onset of the disease.

For administering the effective dose of the compound of the invention to mammals, especially to humans, employable is any suitable administration route. For example, the route may be oral administration, rectal administration, local administration, intravenous administration, ophthalmic administration, lung administration or nasal administration. Examples of the administration forms are tablets, troches, powders, suspensions, solutions, capsules, creams, aerosols. Preferred are oral tablets.

In preparing oral compositions, usable are any ordinary pharmaceutical media. Their examples are water, glycol, oil, alcohol, fragrant additives, preservatives, colorants. In preparing liquid compositions for oral administration, for example, mentioned are suspensions, elixirs and solutions. Their carriers are, for example, starch, sugar, microcrystalline cellulose, diluent, granulating promoter, lubricant, binder, disintegrator. In preparing solid compositions for oral administration, for example, mentioned are powders, capsules and tablets. Above all, such solid compositions for oral administration are preferred.

In view of the easiness in their administration, tablets and capsules are the most advantageous forms for oral administration. If desired, the tablets may be coated according to standard aqueous or non-aqueous coating techniques.

In addition to the above-mentioned ordinary administration modes for them, the compounds of formula (VIII) may also be administered according to controlled release systems and/or controlled delivery systems, for example, as in U.S. Pat. Nos. 3,845,770, 3,916,899, 3,536,809, 3,598,123, 3,630,200 and 4,008,719.

The pharmaceutical composition of the invention suitable for oral administration includes capsules, cashews and tablets that contain a predetermined amount of the active ingredient in the form of powders or granules thereof, or in the form of water-soluble liquids, water-insoluble liquids, oil-in-water emulsions or water-in-oil emulsions thereof. These compositions may be prepared in any pharmaceutical methods, and all the methods include a process of combining the active ingredient with a carrier of one or more necessary ingredients.

In general, the active ingredient is uniformly and fully mixed with a liquid carrier, or a well-separated solid carrier or with both the two, and then, if desired, the product is shaped into suitable forms to prepare the composition. For example, tablets are produced through compression and shaping, optionally along with one or more side components. Using a suitable machine, compressed tablets may be produced by mixing the active ingredient optionally with binder, lubricant, inert vehicle, surfactant or dispersant and compressing the resulting mix in any desired manner into powders or granules.

Shaped tablets may be prepared by shaping a mixture of a powdery wet compound and an inert liquid diluent, using a suitable machine.

Preferably, the tablets each contain from about 1 mg to 1 g of the active ingredient; and the cashews and the capsules each contain from about 1 mg to 500 mg of the active ingredient.

Examples of the administration modes of the compounds of formula (VIII) for pharmaceutical use are as follows:

TABLE 2
Suspension for Injection (I. M.)

| | mg/ml |
|---|---|
| compound of formula (I) | 10 |
| methyl cellulose | 5.0 |
| Tween 80 | 0.5 |
| benzyl alcohol | 9.0 |
| benzalkonium chloride | 1.0 |

water for injection added to make 1.0 ml

TABLE 3

| Tablets | mg/tablet | |
|---|---|---|
| compound of formula (I) | 25 | |
| methyl cellulose | 415 | |
| Tween 80 | 14.0 | |
| benzyl alcohol | 43.5 | |
| magnesium stearate | 2.5 | |
| total | 500 | mg |

TABLE 4

| Capsules | mg/capsule | |
|---|---|---|
| compound of formula (I) | 25 | |
| lactose powder | 573.5 | |
| magnesium stearate | 1.5 | |
| total | 600 | mg |

TABLE 5

| Aerosol | per one container | |
|---|---|---|
| compound of formula (I) | 24 | mg |
| lecithin, NF Liq. Conc. | 1.2 | mg |
| trichlorofluoromethane, NF | 4.025 | g |
| dichlorodifluoromethane, NF | 12.15 | g |

The compounds of formula (VIII) may be used, as combined with any other drugs usable not only for type II diabetes-associated diseases or symptoms but also for remedy/prevention/retardation of the onset of type II diabetes. The additional drugs may be administered in any administration route and dose generally employed in the art, simultaneously with or separately from the compound of formula (VIII).

In case where the compound of formula (VIII) is used along with one or more other drugs, then a pharmaceutical composition comprising the compound of formula (VIII) and the additional drug is preferred. Accordingly, the pharmaceutical composition of the invention may comprise not only the compound of formula (VIII) but also one or more such active ingredients. Examples of the active ingredients that may be combined with the compounds of formula (VIII) are mentioned below, which, however, are not limitative. These may be separately administered or may be administered simultaneously as contained in the same pharmaceutical composition.

(a) other glucokinase activators,
(b) bis-guanides (e.g., buformin, metoformin, fenformin,),
(c) PPAR agonists (e.g., triglytazon, pioglytazon, nosiglytazon),
(d) insulin,
(e) somatostatin,
(f) α-glucosidase inhibitors (e.g., boglybose, miglytol, acarbose),
(g) insulin secretion promoters (e.g., acetohexamide, calbutamide, chlorpropamide, glybomlide, glycrazide, glymerpiride, glypidide, glyquidine, glysoxepide, glyburide, glyhexamide, glypinamide, fenbutamide, trazamide, tolbutamide, tolcyclamide, nateglynide, repaglynide),
(h) DPP-IV (dipeptidyl peptidase IV) inhibitors, and
(i) glucose intake promoters.

The weight ratio of the compound of formula (VIII) to the second active ingredient may vary within a broad range, and depends on the effective amount of the individual active ingredients. Accordingly, for example, when the compound of formula (VIII) is combined with a PPAR agonist, then the weight ratio of the compound of formula (VIII) to the PPAR agonist may be generally from about 1000/1 to 1/1000, preferably from about 200/1 to 1/200. The combination of the compound of formula (VIII) and the other active ingredient may be within the above-mentioned range. In any case, an effective amount of the individual ingredients should be in the combination.

The glucokinase-activating potency of the compounds of formula (I) of the invention and a test method for it are described below.

### Pharmacology Test 1 (Glucokinase-Activating Effect)

The excellent glucokinase-activating effect of the compounds of formula (I) may be determined by a method described in references (for example, Diabetes, Vol. 45, pp. 1671-1677, 1996), or in accordance with it.

The glucokinase activity may be determined not by directly measuring glucose-6-phosphate but by measuring the level of Thio-NADH, which is produced when a reporter enzyme, glucose-6-phosphate dehydrogenase produces phosphogluconolactone from glucose-6-phosphate, and based on the level, the degree of glucokinase activity of the compound tested may be determined.

In this assay, used was a recombinant human liver GK, which was expressed by E. coli as a FLAG fusion protein therein and was purified by ANTIFLAG M2 AFFINITY GEL (Sigma).

Using a flat-bottomed 96-well plate, the assay was carried out at 30° C. 69 μl of an assay buffer (25 mM Hepes Buffer/pH=7.2, 2 mM MgCl₂, 1 mM ATP, 0.5 mM TNAD, 1 mM dithiothreitol) was put into the plate, and 1 μl of a DMSO solution of the compound or DMSO alone as a control was added thereto. Next, 20 μl of an enzyme mixture (FLAG-GK, 20 U/ml G6PDH) cooled in ice was added to it, and 10 μl of a substrate, 25 mM glucose was added to it, and the reaction was initiated (final glucose concentration=2.5 mM).

After the start of the reaction, the increase in the absorbance at 405 nm was measured for 12 minutes at intervals of 30 seconds, and the increase for the first 5 minutes was used for evaluating the compound tested. FLAG-GK was added so that the absorbance increase after 5 minutes in the presence of 1% DMSO could be from 0.04 to 0.06.

The OD level of the DMSO control was set as 100%; and the OD level of the test compound at different concentrations was determined. From the OD level at each concentration, Emax (%) and EC50 (μM) were computed and used as the index of the GK-activating potency of the compound.

Emax (%) of the crystalline of 3-(6-ethanesulfonylpyridin-3-yloxy)-5-((1S)-2-hydroxy-1-methyl-ethoxy)-N-(1-methyl-1H-pyrazol-3-yl)benzamide methanesulfonate was 828 and EC50 (μM) was 0.03.

A salt containing compound (VI) and 1,4-diazabicyclo[2.2.2]octane of 2:1, an alkylsulfonate of compound (VIII), a methanesulfonate of compound (VIII), and a methanesulfonate of a compound expressed by a formula (VIII-1) are novel.

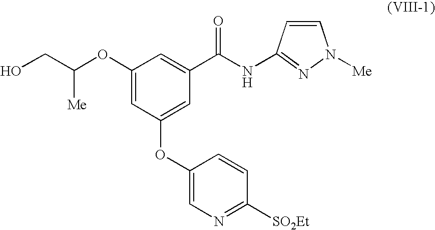

### EXAMPLE

Hereinafter, the present invention will be described further specifically by Example, the present invention is by no means restricted thereby.

### Example 1

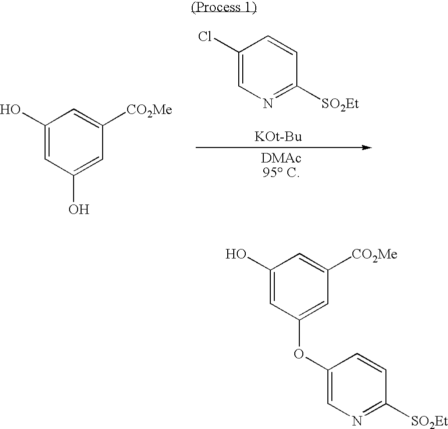

To a vacuum degassed solution of methyl 3,5-dihydroxybenzoate (50.8 g, 0.293 mol) in DMAc (N,N-dimethylacetamide, 280 mL) under N₂ atmosphere was added a solution of t-BuOK (57.7 g, 0.488 mol) in DMAc (400 mL) dropwise at ambient temperature over 2 h. The slurry mixture was stirred for 1 h at ambient temperature, 1 h at 50° C., and 1 h at 95° C. Then, a solution of 5-chloro-2-ethanesulfonylpyridine (40 g, 0.195 mol) in DMAc (120 mL) was added dropwise over 5 h at 9˜100° C. After the addition, the reaction mixture was then aged at 95° C. for 2 h. The reaction mixture was cooled to ambient temperature and poured into 1N aqueous HCl (640 mL) with external cooling to maintain the internal temperature at below 35° C. i-PrOAc (800 mL) was added and the organic phase was separated. The aqueous phase was extracted once with i-PrOAc (600 mL). The combined organic phase was washed with 5% NaCl aqueous solution (3×400 mL). The organic layer was azeotropically dried and concentrated to 240 mL. i-PrOH (24 mL) was added and the solution was seeded with seeds (800 mg). The slurry was allowed to age at ambient temperature for >3 h. n-Heptane (420 mL) was added dropwise over 10 h at ambient temperature.

The resulting slurry was aged for additional 2 h at ambient temperature followed by 2 h at 0˜5° C. before filtration. The wet cake was displacement washed with 30% i-PrOAc in heptane (200 mL) followed by slurry wash with i-PrOAc/heptane (200 mL of 30% i-PrOAc in heptane, then 200 mL of 25% i-PrOAc in heptane). The wet cake was dried under nitrogen in vacuum at 40° C. overnight to give 49.2 g of the desired product (75 wt % yield).

TABLE 6
HPLC conditions:

| Column: | YMC Pack ODS-AM303, 250 mm × 4.6 mm | | |
|---|---|---|---|
| Column temperature: | 40° C. | | |
| Flow rate: | 1.0 mL/min | | |
| Wavelength: | 210 nm | | |
| | min | CH₃CN | 0.1% H₃PO₄ |
| Gradient: | 0 | 50 | 50 |
| | 5 | 50 | 50 |
| | 15 | 90 | 10 |

| | |
|---|---|
| Retention times: | |
| Benzoate | 3.2 min |
| Chloro-sulfonylpyridine | 4.5 min |
| Product | 5.2 min |

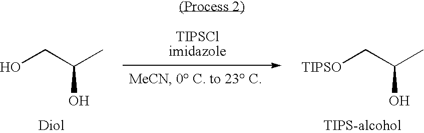

To a solution of (R)-1,2-propanediol (100 g, 1.314 mol) and imidazole (116 g, 1.708 mol) in acetonitrile (600 mL) at 0° C. was added triisopropylchlorosilane (TIPSCl, 266 g, 1.380 mol) dropwise over 3 h while maintaining the batch temperature at 0-5° C. The resulting slurry was stirred for additional 1 h at 0-5° C. and 3 h at ambient temperature. The reaction was quenched by addition of 15% NaCl aqueous solution (1 L) and toluene (800 mL). The organic layer was separated and washed with 15% NaCl aqueous solution (500 mL). 282.8 g of the desired product (93% yield GC assay).

### GC Method

| | |
|---|---|
| Column: | Agilent 19091Z-413E, 30 m × 0.32 mm × 0.25 um |
| Const flow | 1.5 mL/min |
| Oven temp: | 60° C., hold 2 min, ramp 25° C./min to 220° C. then |
| | 40° C./min to 280° C. |
| Retention times: | |
| 2.85 min | (R)-1,2-propanediol |
| 6.06 min | TIPSCl |
| Product | 7.56 min |

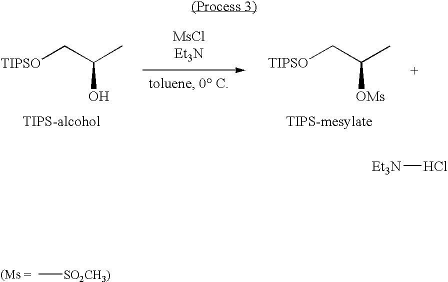

The above crude solution of TIPS-alcohol (282.8 g assay, 1.217 mol) was azetropically concentrated to ˜500 mL and diluted with toluene to 3.1 L. The resulting solution was cooled to 0° C., and triethylamine (172.0 g, 1.703 mol) was charged. Methanesulfonyl chloride (167.0 g, 1.460 mol) was added dropwise over 2 h while maintaining the batch temperature at 0-3° C. The resulting slurry was stirred at 0-5° C. for 1 h. The reaction was then quenched by addition of H₂O (1.7 L) and the resulting mixture was allowed to warm to ambient temperature. The organic layer was washed with H₂O (850 mL). The resulting hazy solution was azetropically concentrated to about 600 mL, which was filtered and used directly for the next step. (60.6 wt %, 94% yield).

### GC Method

| | |
|---|---|
| Column: | Agilent 19091Z-413E, 30 m × 0.32 mm × 0.25 um |
| Const flow | 1.5 mL/min |
| Oven temp: | 60° C., hold 2 min, ramp 25° C./min to 220° C. then |
| | 40° C./min to 280° C. |
| Retention times: | |
| 3.18 min | Toluene |
| 7.56 min | TIPS-alcohol |
| 9.48 min | TIPS-mesylate (desired product) |

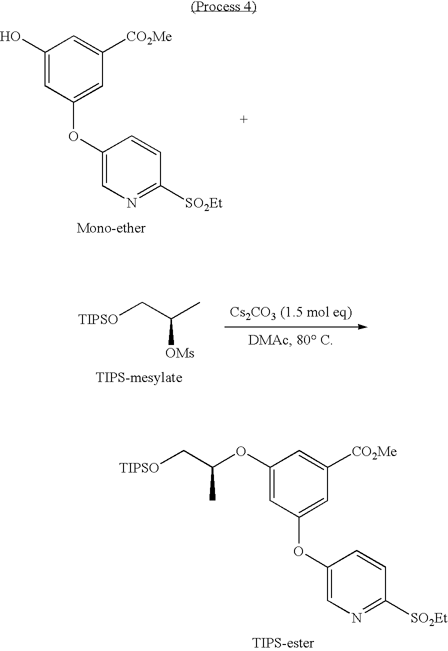

A crude solution of TIPS-mesylate (19.57 g crude, 60.6 wt %, 11.86 g assay, 38.2 mmol) was diluted with dry DMAc (60 mL). Cesium carbonate (powdered, 14.36 g, 44.1 mmol) followed by the mono-ether intermediate (10.41 g crude, 95.1 wt %, 9.91 g assay, 29.4 mmol) were charged while maintaining a vigorous agitation. The reaction mixture was stirred at 80° C. until the reaction was deemed complete (about 8-12 h). The reaction was cooled to 0° C. and diluted with MTBE (79 mL). H2O (39.6 mL) was charged slowly while maintaining the batch temperature below 20° C. After the solution was warmed to ambient temperature, the aqueous layer was discarded. The organic layer was directly used for the next step. HPLC assay of organic layer: 15.15 g, 93% yield.

TABLE 7
HPLC conditions:

| Column: | YMC Pack AS-303, 250 mm × 4.6 mm | | |
|---|---|---|---|
| Column temperature: | 25° C. | | |
| Flow rate: | 1.0 mL/min | | |
| Wavelength: | 220 nm | | |
| Gradient: | min | CH₃CN | 0.1% H₃PO₄ |
| | 0 | 50 | 50 |
| | 5 | 70 | 30 |
| | 7 | 95 | 5 |
| | 24 | 95 | 5 |

Retention times:

| | |
|---|---|
| Methyl 3,5-dihydroxybenzoate | 3.5 min |
| OH-acid | 3.8 min |
| Mono-ether (starting material) | 5.4 min |
| OH-ester | 5.6 min |
| Toluene | 9.5 min |
| TIPS-acid | 14.0 min |
| Product (TIPS-ester) | 17.7 min |

OH-ester 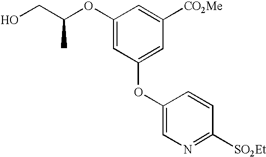
TIPS-acid 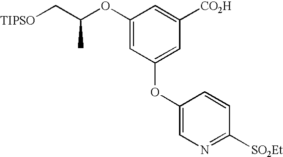
OH-acid

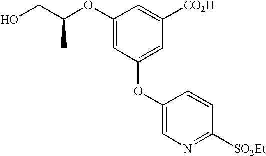

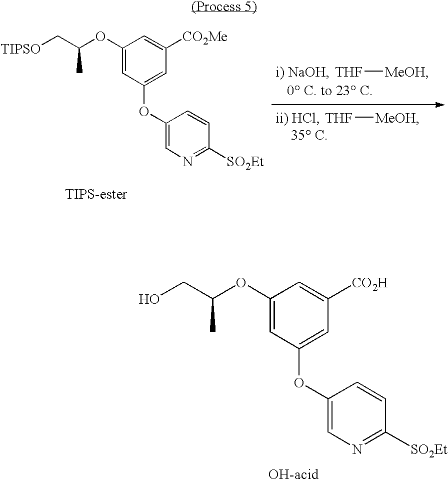

The crude solution of TIPS-ester (9.70 g assay, 17.58 mmol) was solvent-switched to THF (Final volume: ˜57 mL). MeOH (19.40 mL) was added and the batch was cooled to 0° C. Aqueous NaOH (5 N, 5.63 mL, 28.10 mmol) was added dropwise while maintaining the internal temperature below 5° C. The resulting solution was aged for 1 h at 0-5° C. followed by 6 h at ambient temperature. Aqueous HCl (17.58 mL, 4 M, 70.30 mmol, 4.0 mol eq) was then charged. The resulting hazy solution was heated to 35° C. for 6-8 h. The reaction was allowed to cool to ambient temperature and i-PrOAc (48.5 mL) and 15% NaCl aqueous solution (24.3 mL) were added. The aqueous layer was separated and extracted with i-PrOAc (24.3 mL). The combined organic layer was washed with 15% NaCl aqueous solution (48.5 mL). HPLC assay: 6.81 g.

HPLC conditions are same as the previous step. Retention time of product (OH-acid): 14.0 min.

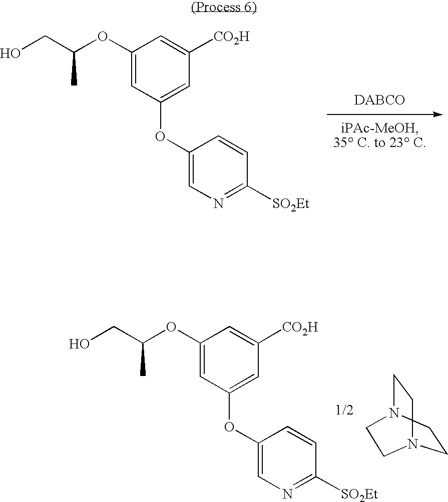

A crude solution of the compound 7-2 (40.9 g assay, 107.2 mmol) was concentrated to 300 mL and azeotropically dried with i-PrOAc. The solution was filtered to remove a small amount of inorganic salt and diluted with i-PrOAc to 370 mL. MeOH (61.4 mL) was added and the reaction solution was heated to 50° C. A solution of DABCO (7.82 g, 69.68 mmol) in i-PrOAc (164 mL) was prepared in a separate flask. A portion of this DABCO solution (24.6 mL) was charged to the solution of the OH-acid. A well-dispersed slurry of DABCO salt (818 mg) in i-PrOAc (8.18 mL) was added, and the slurry was stirred at 50° C. for 2 h to form a seed bed. Then, the remaining DABCO solution was charged at 50° C. over 6 h. The slurry was aged at 50° C. for 1 h and allowed to cool to ambient temperature over 1 h, followed by aging at ambient temperature for 5 h. The solid was collected by filtration, and washed with 5% MeOH/1-PrOAc (82 mL, displacement wash) and i-PrOAc (280 mL, slurry wash). The wet cake was suction-dried to afford the DABCO salt (45.61 g) as off-white solid. 98.0 wt %.

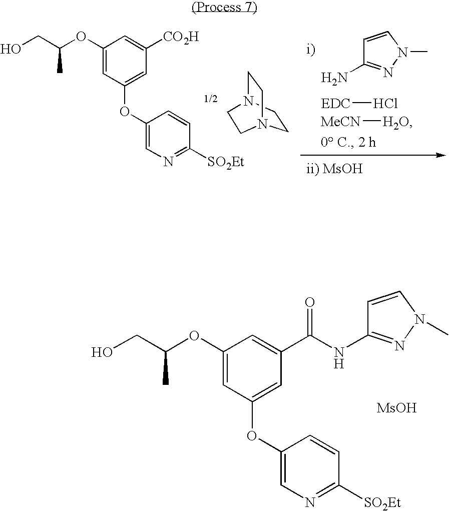

To a solution of DABCO salt 8 (20.0 g, 45.72 mmol) in MeCN (120 mL) and H₂O (80 mL) at 0-5° C. were added pyridine (1.08 g, 13.72 mmol) and 3-amino-1-methylpyrazole (5.33 g, 54.86 mmol). EDC-HCl (10.52 g, 54.86 mmol) was charged, and the reaction solution was aged at 0-5° C. for 30 min. 1 N aqueous HCl (45.72 mL, 45.72 mmol) was added dropwise at 0-5° C. over 3 h. The biphasic solution was stirred at 0-5° C. for 4 h. The reaction was allowed to warm to ambient temperature and diluted with i-PrOAc (160 mL), H₂O (140 mL) and 1 M HCl (13.72 mL). The aqueous layer was separated and extracted with i-PrOAc (160 mL). The combined organic layer was washed with 2% citiric acid/20% NaCl aqueous (100 mL) and 25% NaCl (100 mL). The solution was azeotropically dried with MeCN. The resulting mixture was filtered to remove inorganic salt and diluted with MeCN to give a compound 9-1 (24 wt %).

The solution was diluted with toluene (80 mL) and heated to 30° C., followed by charging methanesulfonic acid (1.10 g, 11.43 mmol). The resulting solution was seeded with 1.27 g of MsOH salt. After 2 h at 25-35° C., a solution of methanesulfonic acid (3.73 g, 38.86 mmol) in MeCN (40 mL) and toluene (40 mL) was added dropwise at 25-35° C. over 12 h. The resulting slurry was stirred at 25-35° C. for 1 h, and allowed to cool to 5-10° C. over 2 h, followed by stirring at 5-10° C. for 6 h. The wet cake was washed with 1:1 MeCN/toluene (80 mL, displacement wash, 5-10° C.), 1:9 MeCN/toluene (80 mL, displacement wash, ambient temperature) and MTBE (160 mL, displacement wash, ambient temperature) and dried in vacuum oven (45° C.) to afford 3-(6-ethanesulfonylpyridin-3-yloxy)-5-((1S)-2-hydroxy-1-methyl-ethoxy)-N-(1-methyl-1H-pyrazol-3-yl)benzamide methanesulfonate 9 as off-white solid. 90% yield.

TABLE 8
HPLC conditions:

| Column: | YMC-Pack ProC18, 150 × 4.6 mm, 3 um | | |
|---|---|---|---|
| | particles | | |
| Column temperature: | 40° C. | | |
| Flow rate: | 1.0 mL/min | | |
| Run time: | 25 min | | |
| Wavelength: | 220 nm | | |
| | min | CH₃CN | 0.1% H₃PO₄ |
| Gradient: | 0 | 20 | 80 |
| | 4 | 35 | 65 |
| | 9 | 35 | 65 |
| | 18 | 60 | 40 |
| | 19 | 85 | 15 |
| | 25 | 85 | 15 |

Retention times:

| Component | RRT |
|---|---|
| Pyridine | 0.15 |
| 3-amino-1-methylpyrazole | 0.16 |
| DABCO salt | 0.89 |
| product | 1.00 |

Another procedure of production of 3-(6-ethanesulfonylpyridin-3-yloxy)-5-((1S)-2-hydroxy-1-methyl-ethoxy)-N-(1-methyl-1H-pyrazol-3-yl)benzamide methanesulfonate was described in Example 2.

### Example 2

### Production of 3-(6-ethanesulfonylpyridin-3-yloxy)-5-((1S)-2-hydroxy-1-methyl-ethoxy)-N-(1-methyl-1H-pyrazol-3-yl)benzamide methanesulfonate

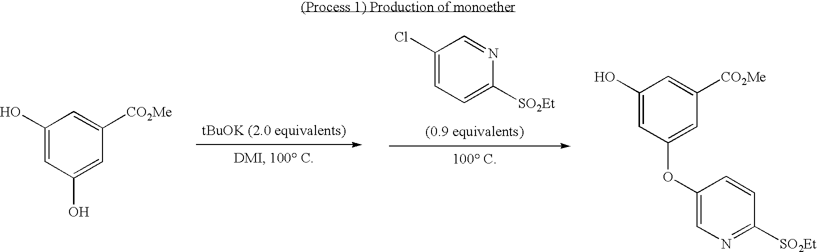

Methyl 3,5-dihdroxybenzoate (2.4 kg) was dissolved in 1,3-dimethyl-2-imidazolidinone (72 L) at room temperature. To this solution, potassium tert-butoxide (3.20 kg) was added below 50° C. This suspension was heated to 100° C. and aged under diminished pressure (20 Torr) at 100° C. for 30 minutes while removing tert-butyl alcohol by distillation.

To this suspension, 1,3-dimethyl-2-imidazolidinone solution (9.6 L) of 3-chloro-6-(ethanesulfonyl)pyridine (2.64 kg) was added dropwise over 6 hours or more at 95-100° C. After adding dropwise, this suspension was aged at 100° C. for more than 2 hours. The end of reaction was checked by HPLC.

Next, this suspension was cooled to room temperature. To this suspension, 1N hydrochloric acid (14.3 L) and deionized water (96 L) were added at 20-30° C.

The resulting solution was washed twice with a mixed solution of isopropyl acetate (12 L) and heptane (36 L). The product was extracted from 1,3-dimethyl-2-imidazolidinone-deionized water layer three times with a mixed solution of isopropyl acetate (43.2 L) and heptane (4.8 L). The organic layers combined were washed with a tetraborate buffer solution (pH=9, 12 L) and three times with deionized water (48 L).

The organic layer was dehydrated at 20 Torr, 40° C. by azeotropic distillation to convert the solvent into 1,3-dimethyl-2-imidazolidinone (60 L). Monoether body 3 of 2.81 g was obtained as a 1,3-dimethyl-2-imidazolidinone solution.

Azeotropic distillation was continued until KF became below 500 ppm.

Conversion of solvent was confirmed by GC.

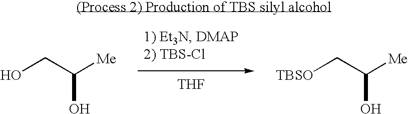

To a 50 L container equipped with a mechanical stirrer, thermocouple probe and nitrogen inlet, were added (2R)-1,2-dihydroxypropane (3.00 kg), triethylamine (6.04 L), dimethylaminopyridine (241 g) and THF (30 L). After the solution was cooled below 5° C., TBS-Cl (6.24 kg) was added thereto over 3 hours or more. The homogeneous solution became an inhomogeneous suspension. The resulting suspension was stirred below 5° C. for 30 minutes, subsequently at room temperature for 17 hours.

The consumption of raw material was confirmed by TLC (heptane/ethyl acetate: 1/1). After 1,2-dihydropropane was completely consumed, water (15 L) was added. The organic layer was separated, and washed with 20% saline (10 L). The organic layer was concentrated under diminished pressure. The coarse residue produced was distilled (10 Torr, 77-80° C.).

The yield was 5.25 kg and 5.63 kg (75%, 99.85% ee 2nd batch).

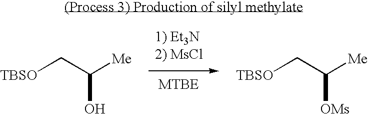

To a 150 L flask equipped with a mechanical stirrer, thermocouple probe and nitrogen inlet, were added silyl alcohol body (4.69 kg), triethylamine (2.86 kg) and MTBE (methyl tertiary butyl ether; 33.15 L) dehydrated at 200 ppm using 4A molecular sieve before usage.

After the resulting solution was cooled at 5° C., methanesulfonyl chloride (2.97 kg) was added thereto over 50 minutes or more. The homogeneous solution was rapidly changed to an inhomogeneous solution. The resulting solution was stirred below 5° C. for 1 hour, subsequently stirred at room temperature for 1 hour.

The consumption of alcohol body of raw material was confirmed by TLC (toluene/ethyl acetate: 5/1). After alcohol body was completely consumed, water (22.40 kg) was added. The organic layer was separated, and washed with water (13.44 kg) and 20% saline (13.44 L). By azeotropy of MTME (68 kg) at 800 ppm or less, the amount of water was reduced (first time 609.3 ppm, second time 718.0 ppm), subsequently, the solvent was converted into DMI (38 kg).

The ratio DMI/MTBE was determined to be 93/7 by GC. The chemical yield was quantitative by HPLC assay of organic layer.

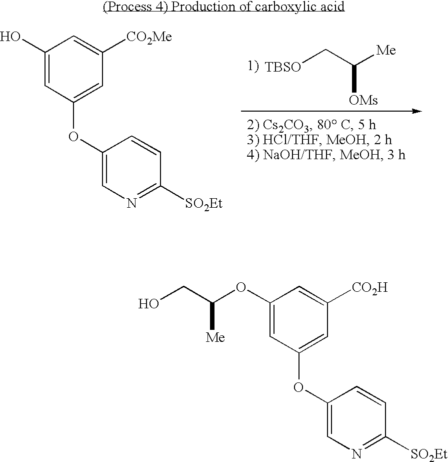

To a 150 L container equipped with a mechanical stirrer, thermocouple probe and nitrogen inlet, DMI solution of silyl methylate (6.34 kg) and DMI solution of monoether body (7.97 kg) were added. After vacuum deairing for 10 minutes, the amount of water was measured (1st batch 519.9 ppm, 2nd batch 609.3 ppm). Cesium carbonate (7.67 kg) was added thereto, the resulting mixture was heated at 80° C. After being kept at 80° C. for 5 hours, the reaction mixture was cooled to room temperature.

The resulting mixture was divided by half. One part was kept under nitrogen atmosphere at room temperature for 2 days. The other part was quenched with water (36 kg) while maintaining the temperature below 30° C. The mixture produced was extracted with isopropyl acetate (first time 31.64 kg, second time 15.73 kg, third time 7.87 kg). The organic layers combined were washed with water (3×36 kg). The organic layer was concentrated under diminished pressure. The coarse residue produced was dissolved in THF (5 L) and re-concentrated under diminished pressure.

The coarse residue produced was dissolved in THF (19.2 kg) and MeOH (1.8 L). While maintaining the temperature below 10° C., 2N hydrochloric acid (5.4 L) was added thereto, and stirred at room temperature for 2 hours. While maintaining the temperature below 10° C., MeOH (9.0 L) and 5N sodium hydroxide (5.4 L) were added, and stirred at room temperature for 3 hours.

After water (27.0 kg) was added, the resulting solution was washed with heptane (2×12.31 kg) and MTBE (2×12.32 kg). While stirring vigorously, MTBE (13.32 kg) was added thereto, pH of the solution was adjusted to 2.0-3.0. The aqueous layer was extracted with MTBE (13.32 kg). The organic layers combined were washed with water (9.0 kg) and 20% saline (9.0 L).

The chemical yield was determined by HPLC assay of the two solutions obtained to be 92% for the first batch and 91% for the second batch.

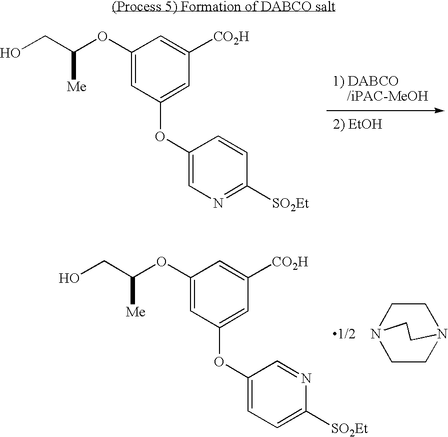

MTBE solution of the above carboxylic acid 7 was transferred to an evaporator N-100. After the whole solvent was distilled away under diminished pressure, isopropyl acetate (66.07 kg) was added, and concentrated to 25.2 L. The amount of water in isopropyl acetate solution was 540 ppm or less, and it was confirmed (by GC) that isopropyl acetate solution contained 0.1% or less of residual MTBE.

The reaction mixture was transferred to a container V-245-1, washed with isopropyl acetate that had been passed through a filter (1 mmφ), and methanol (7.12 kg) was added thereto.

The reaction mixture was heated at 50° C. Isopropyl acetate (2.26 kg) of DABCO (0.10 kg) and seed crystal (36 g) were added thereto. After being aged at 50-55° C. for 1 hour, to the solution, DABCO (0.62 kg)/isopropyl acetate (13.47 kg) was added over 3 hours, and the drip rate was controlled by a needle valve. As colorless slurry began forming, stirring became difficult. This slurry was stirred at 50° C. for 9 hours, 40° C. for 2 hours, at room temperature all night, and aged.

After all-night aging, the slurry was stirred until the concentration of supernatant was 7 mg/ml or less, and the temperature of the mixture became 25° C.

The slurry formed was filtered using Filter Pot (FF15). The residual slurry was rinsed with mother liquid. The cake was washed with isopropyl acetate-methanol (19:1, 15.66 kg) and isopropyl acetate (15.73 kg), and dried at 50° C. under diminished pressure all night. The residual solvent was MTBE, isopropyl acetate, methanol (<0.5%) and water (KF<1%).

### Recrystallization

The DABCO salt obtained was put in a container (V-245-1), and ethanol (48.24 kg) was poured thereto. All crystals were completely melted at 65° C., subsequently cooled to 50° C. Seed crystal was added at 50° C., and stirred. After aging for 3 hours, the slurry was cooled to room temperature.

After aging all night, the crystal was washed with cool ethanol (below 5° C., 9.48 kg), and dried at 50° C. under diminished pressure.

The residual solvent was ethanol, isopropyl acetate, methanol (<0.5%), MTBE (<0.1%), and water (KF<1%).

DABCO salt was obtained as a colorless crystal of 3.099 kg (96.98 area %, first time) and 3.6612 kg (98.53 area %, second time).

(The yields from methyl 3,5-dihydroxybenzoate were 33% and 39%, respectively).

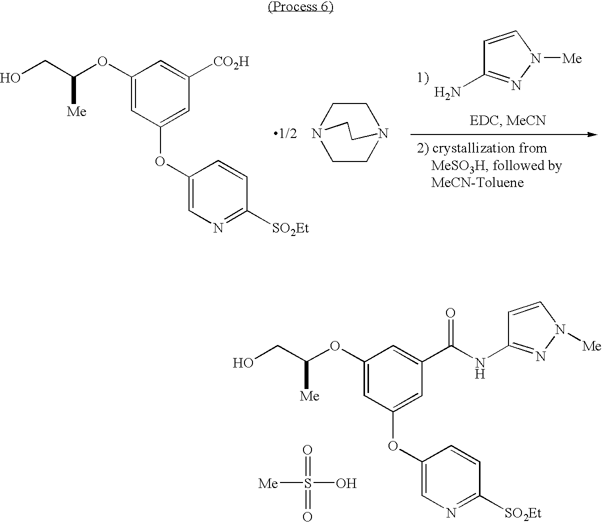

To a 150 L container equipped with a mechanical stirrer, thermocouple probe and nitrogen inlet, DABCO salt (6.50 kg), MTBE (97.5 L) and 1N hydrochloric acid (13.0 L) were added.

Two layers were vigorously mixed until all solids were dissolved to separate into layers. The organic layer was converted from MTBE to acetonitrile solution of about 39 L.

While stirring the batch at 20° C. to 30° C. for 2 hours, acetonitrile solution of free carboxylic acid produced was put to a container together with water (32.5 L), 3-amino-1-methylpyrazole (1.73 kg), pyridine (1.18 L) and EDC-hydrochloride (3.42 kg). The consumption of carboxylic acid of raw material was monitored by HPLC.

The batch was quenched with 1N hydrochloric acid of 13 L and MTBE of 32.5 L. After mixing, two layers were separated. The aqueous layer was extracted twice with a fresh MTBE (32.5 L). The organic layers combined were washed with 15% saline (26 L), subsequently, washed twice with 1N sodium carbonate aqueous solution (26 L). After area % of a target was checked by HPLC, the aqueous layer was discarded.

The MTBE layer was concentrated, the solvent was converted into acetonitrile of about 39.1 L. The acetonitrile solution was filtered through a filter of 1.0 μL, and washed with 32.5 L of toluene.

To the acetonitrile/toluene solution, 19.5 L of half of acetronitrile mixture, 52 L of toluene and 1.2 L of methanesulfonic acid were slowly added over 1 hour or more while being maintained at 47 to 50° C. After 32.5 g of seed crystal was added thereto, the mixture was aged at 50° C. for 1 hour.

After the batch was completely crystallized, the remaining mixture of acetonitrile solution, toluene and methanesulfonic acid were added to the slurry over 1 hour or more while being maintained at 47 to 50° C. Subsequently, the slurry was aged at 50° C. for 2 hours, and slowly cooled to room temperature over 10 hours or more, then aged all night at room temperature.

The crystal was filtered, and washed with 45.5 L of toluene/acetonitrile (9:1), and dried by nitrogen flow, then dried at 60° C. under diminished pressure, thereby to obtain methanesulfonate 9 of 6.64 kg (80.3%) as a colorless crystal.

The loss due to mother liquid and washing was 1.26 kg in total.

Seed crystal of 3-(6-ethanesulfonylpyridin-3-yloxy)-5-((1S)-2-hydroxy-1-methyl-ethoxy)-N-(1-methyl-1H-pyrazol-3-yl)benzamide methanesulfonate used in the recrystallization was obtained as follow; amorphous of 3-(6-ethanesulfonylpyridin-3-yloxy)-5-((1S)-2-hydroxy-1-methyl-ethoxy)-N-(1-methyl-1H-pyrazol-3-yl)benzamide methanesulfonate (200 mg) obtained according to the procedure described in example 117 of WO2004/076420 was dissolved in ethyl acetate (10 mL), and to the solution methanesulfonic acid (31 μL) in ethyl acetate (310 μL) was added. The reaction mixture was stirred for 2 min and was left for 18 hours followed by filtration of resulting solid, methanesulfonate (170 mg) was obtained as a white solid.

Various analyses were conducted for 3-(6-ethanesulfonylpyridin-3-yloxy)-5-((1S)-2-hydroxy-1-methyl-ethoxy)-N-(1-methyl-1H-pyrazol-3-yl)benzamide methanesulfonate, and the result are shown as follows.

### (1) Thermo Analysis

### 1. Thermogravimetric Analysis

A Perkin Elmer model TG 7 or equivalent instrument is used. Experiments are performed under a flow of nitrogen and using a heating rate 10° C./min to a maximum temperature of 500° C. Approximately 10 mg of sample is added to the platinum pan. Weight/temperature data are collected automatically by the instrument. Compound 9 doesn't show obvious weight loss until 180° C.

### 2. Differential Scanning Calorimetry

Approximately 2 mg of sample is accurately weighed into an aluminum DSC pan, and the pan is subsequently sealed under nitrogen atmosphere. The sample is then analyzed by differential scanning calorimetry using a TA Instruments or equivalent at a heating rate of 10° C./min. from approximately 25° C. to 350° C. After 2 mg of compound were measured, report the onset temperature, peak temperature and enthalpy of the melting endotherm. As a result, compound 9 has Tonset at 137° C. and T max at 140° C. and heat of fusion is (ΔH) 106 J/g.

### (2). X-Ray Powder Diffraction

Obtain the X-ray diffraction pattern of a finely-powdered, randomly orientated sample, using copper K alpha X-radiation from 4 to 40 theta.

Bruker powder X-ray diffractometer D8ADVANCE (at 2 kW) (bull car ray X id bull car ray X id Co., Ltd.) is used, and a powder X-ray diffraction experiment was done. Measurement condition is as table 9, Diffraction angle 2θ and the intensity are shown in table 10.

TABLE 9

| | |
|---|---|
| Start Position [° 2Th.] | 4 |
| End Position [° 2Th.] | 40 |
| Step Size [° 2Th.] | 0.014 |
| Scanning Step Time [s] | 42.4 |
| Scan Type | continuous |
| Diffusion Slit (DS) Size [°] | 0.1 |
| Sample Width [mm] | 10.00 |
| Test Temperature [° C.] | 25.00 |
| Target | Cu |
| X-ray Output Set | 35 kV, 40 mA |
| Goniometer Radius [mm] | 250.00 |
| Incident Side Monochromete | none |

TABLE 10

| 2 | Relative Intensity | 2 | Relative Intensity [cps] |
|---|---|---|---|
| 7.9 | 7 | 21.8 | 20 |
| 9.6 | 49 | 22.2 | 16 |
| 11.0 | 14 | 22.5 | 13 |
| 11.8 | 27 | 23.7 | 25 |
| 12.2 | 14 | 24.1 | 12 |
| 13.6 | 10 | 26.1 | 13 |
| 15.2 | 10 | 26.6 | 10 |
| 17.0 | 13 | 27.0 | 8 |
| 17.8 | 18 | 27.3 | 9 |
| 18.2 | 18 | 28.2 | 11 |
| 18.8 | 100 | 30.7 | 8 |
| 19.2 | 40 | | |
| 19.7 | 27 | | |
| 20.3 | 21 | | |
| 21.3 | 23 | | |

Note)
The intensity is a relative value based on the maximum, 100.

From the result of above thermogravimetric Analysis and X-Ray powder diffraction, 3-(6-ethanesulfonylpyridin-3-yloxy)-5-((1S)-2-hydroxy-1-methyl-ethoxy)-N-(1-methyl-1H-pyrazol-3-yl)benzamide methanesulfonate is easy to handle, less hygroscopicity, and also stability physically compared to an amorphous form of 3-(6-ethanesulfonylpyridin-3-yloxy)-5-((1S)-2-hydroxy-1-methyl-ethoxy)-N-(1-methyl-1H-pyrazol-3-yl)benzamide disclosed in patent document 1.

According to the present method, alkyl sulfonate of the compound (VIII), especially 3-(6-ethanesulfonylpyridin-3-yloxy)-5-((1S)-2-hydroxy-1-methyl-ethoxy)-N-(1-methyl-1H-pyrazol-3-yl)benzamide methanesulfonate can be provided as a crystal form, and the present method is useful as a process for producing active ingredient for the oral pharmaceutical formulation which can be satisfied in the respect of purity and stability.

### BRIEF DESCRIPTION OF THE DRAWING

FIG. 1 shows a diffraction pattern of a crystal of 3-(6-ethanesulfonylpyridin-3-yloxy)-5-((1S)-2-hydroxy-1-methyl-ethoxy)-N-(1-methyl-1H-pyrazol-3-yl)benzamide methanesulfonate in powdery X-ray diffractiometry.

FIG. 2 shows a IR spectrum of a crystal of 3-(6-ethanesulfonylpyridin-3-yloxy)-5-((1S)-2-hydroxy-1-methyl-ethoxy)-N-(1-methyl-1H-pyrazol-3-yl)benzamide methanesulfonate.

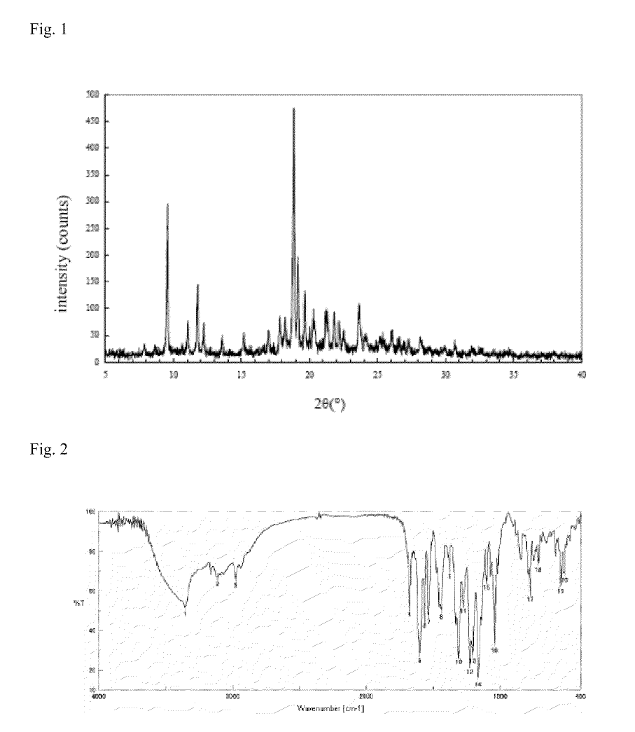

1-17. (canceled)

18. A method for producing a compound expressed by a formula (VIII) or a pharmaceutically acceptable salt thereof, comprising the steps of:
reacting a compound expressed by formula (I) in the presence of a compound of formula (I) and base, 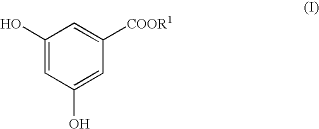
wherein R¹ represents a C₁₋₆ lower alkyl group, 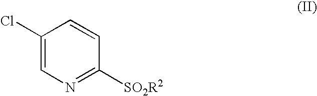
wherein R² represents a C₁₋₆ lower alkyl group to produce a compound of formula (III);
reacting a compound of formula (III) with a compound of formula (IV) in the presence of base, 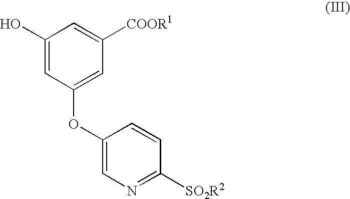
wherein R¹ and R² have the same meaning as described above, 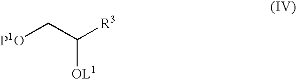
wherein P¹ represents a protective group of a hydroxyl group, R³ represents a C₁₋₆ lower alkyl group, and OL¹ represents a leaving group to produce a compound of formula (V);
removing the protective group P¹ of a hydroxyl group and the protecting group R¹ of a carboxyl group in the compound expressed by formula (V), 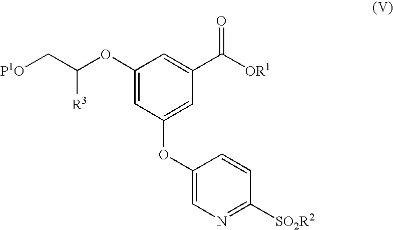
wherein R¹, R², R³ and P¹ have the same meaning as described above to produce a compound of formula (VI);
reacting a compound expressed by formula (VI) with an amine, 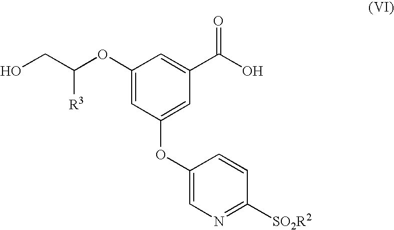
wherein R² and R³ have the same meaning as described above to produce an amine salt;
condensing said salt with a primary amine compound of formula (VII), 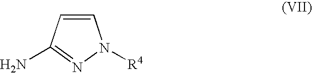
wherein R⁴ represents a C₁₋₆ lower alkyl group, to produce a compound of formula (VIII), 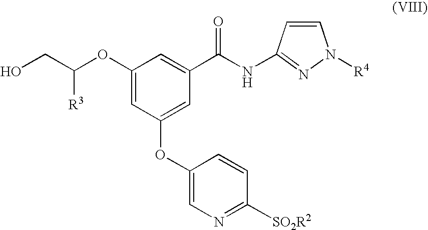
wherein R², R³ and R⁴ have the same meaning as described above.

19. A process for producing a compound expressed by a formula (VIII) 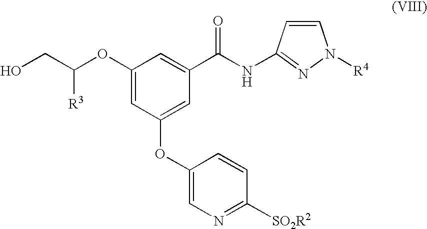
wherein R², R³ and R⁴ have the same meaning as described above or a pharmaceutically acceptable salt thereof,
comprising:
condensing a salt of a compound of formula (VI) and a primary amine compound of formula (VII). 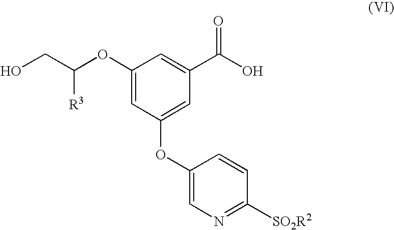
wherein R² and R³ represent a lower alkyl group, 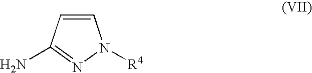
wherein R⁴ represents a lower alkyl group to produce a compound of formula (VIII).

20. A process in accordance with claim 19, wherein the pharmaceutically acceptable salt of a compound expressed by the formula (VIII) is the methanesulfonate.

21. A process in accordance with claim 20, wherein the primary amine compound is 1,4-diazabicyclo[2.2.2]octane.

22. A process in accordance with claim 21, wherein R² is an ethyl group and R³ and R⁴ are methyl groups.

23. A 1,4-diazabicyclo[2.2.2]octane salt of a compound expressed by a formula (VI):

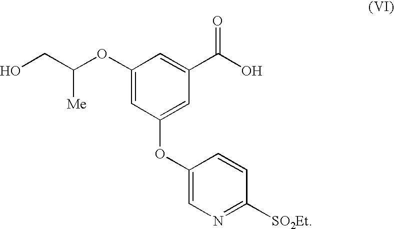

24. A compound of formula (VIII): 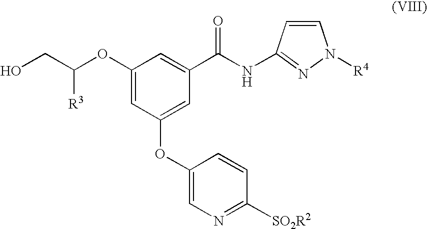
wherein R², R³ and R⁴ represent C₁₋₆ lower alkyl groups.

25. An alkylsulfonate of a compound expressed by a formula (VIII) in accordance with claim 24 in the form of the methanesulfonate.

26. A methanesulfonate of a compound expressed by a formula (VIII-1) in accordance with claim 25:

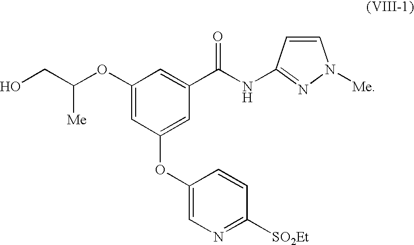

27. A crystalline form of 3-(6-ethanesulfonylpyridin-3-yloxy)-5-((1S)-2-hydroxy-1-methyl-ethoxy)-N-(1-methyl-1H-pyrazol-3-yl)benzamide methanesulfonate in accordance with claim 26.

28. A crystalline form of 3-(6-ethanesulfonylpyridin-3-yloxy)-5-((1S)-2-hydroxy-1-methyl-ethoxy)-N-(1-methyl-1H-pyrazol-3-yl)benzamide methanesulfonate in accordance with claim 26 having main peaks at around 9.6, 11.8, 18.8, 19.2, 19.7, 20.3, 21.3, 21.8 and 23.7 in terms of 2θ(°) in the powder X-ray diffraction pattern.

29. A crystalline form of 3-(6-ethanesulfonylpyridin-3-yloxy)-5-((1S)-2-hydroxy-1-methyl-ethoxy)-N-(1-methyl-1H-pyrazol-3-yl)benzamide methanesulfonate in accordance with claim 26 having T onset at 137° C. and T max at 140° C. and heat of fusion is 106 J/g in the DSC analysis.

30. A crystalline form of 3-(6-ethanesulfonylpyridin-3-yloxy)-5-((1S)-2-hydroxy-1-methyl-ethoxy)-N-(1-methyl-1H-pyrazol-3-yl)benzamide methanesulfonate in accordance with claim 26 having main peaks at around 9.6, 11.8, 18.8, 19.2, 19.7, 20.3, 21.3, 21.8 and 23.7 in terms of 2θ(°) in the powder X-ray diffraction and having T onset at 137° C. and T max at 140° C. and heat of fusion is 106 J/g in the DSC analysis.

31. A crystalline form according to claim 26, characterized by the following absorptions in the FT-IR spectrum (KBr pellet-transmission method) 3355, 3112, 1602, 1567, 1311, 1225, 1205, 1164 and 779 cm⁻¹.

32. A pharmaceutical composition comprising the compound of claim 26 in combination with a pharmaceutically acceptable carrier.
